(19) United States
(12) Patent Application Publication
(10) Pub. No.: US 2024/0100277 A1
(43) Pub. Date: Mar. 28, 2024

(54) DETECTION OF ASYNCHRONIES DURING RESPIRATORY TREATMENT

(71) Applicant: Loewenstein Medical Technology S.A., Luxembourg (LU)

(72) Inventors: Jan VERHOEVEN, Ettingen (DE); Matthias SCHWAIBOLD, Karlsruhe (DE)

(21) Appl. No.: 18/038,361

(22) PCT Filed: Nov. 19, 2021
(86) PCT No.: PCT/EP2021/025452

§ 371 Date: May 23, 2023

(30) Foreign Application Priority Data
Nov. 24, 2020 (DE) 102020007180.5
Aug. 7, 2021 (DE) 102021004081.3

(51) Int. Cl. A61M 16/00

(52) U.S. Cl. CPC A61M 16/026; A61M 16/0003; A61M 2205/18; A61M 2205/502; A61M 2230/42

(57) ABSTRACT

The invention relates to a system for detecting asynchronies between a ventilator and a living being connected to the ventilator, and to a corresponding, partly automatic control of a ventilator. The system for detecting asynchronies between a ventilator and a living being comprises at least one ventilator, the ventilator comprising at least a sensor unit, a processing unit, an arithmetical unit, a detection unit, a memory unit, a monitoring unit, a control unit and a fan-valve unit, and the detection unit detects asynchronies between the ventilator and the living being based on breathing parameters of the living being.

The invention relates to a system for detecting asynchronies between a ventilator and a living being connected to the ventilator as well as a partially automatic control of a ventilator related thereto.

Humans inhale and exhale approximately 20,000 times a day. It is a not insignificant task for the control of a ventilator to detect these breaths correctly and to trigger a respiratory assistance at the correct point in time. Precise algorithms for triggering the breaths are decisive in this case for the success of the treatment and the well-being of the patient/the user. The sensitivity for triggering the breaths is often set manually by an attending physician or carer. The settings thus selected can sometimes not be adapted optimally to the individual requirements of the patient during treatment, however, since the required trigger sensitivity changes or can change due to position changes, various sleep phases, etc.

The object of the present invention is therefore to provide a system for effective and safe respiratory treatment of a living being. The object is achieved by the system according to the invention as claimed in claim 1 and the method as claimed in claim 37.

A system for detecting asynchrony between a ventilator and a living being, comprising at least one ventilator, wherein the at least one ventilator comprises at least
a sensor unit
a preparation unit
a calculation unit
a detection unit
a storage unit
a monitoring unit
a control unit and
a fan/valve unit,
   wherein the detection unit detects asynchronies between the ventilator and the living being on the basis of respiratory parameters of the living being.

In some embodiments of the system, the respiratory parameters include at least pressure and/or flow and/or are determined therefrom.

In some embodiments of the system, the control unit controls the fan/valve unit on the basis of the asynchrony detected by the detection unit.

In some embodiments of the system, the detection of the asynchrony is carried out during the use of the ventilator by the living being. The asynchrony detection is thus carried out while the living being uses the ventilator.

In some embodiments of the system, the detection unit detects missed breaths and short trigger delays.

In some embodiments of the system, the detection unit assesses the short trigger delays and the missed breaths as asynchrony.

In some embodiments of the system, the detection unit detects the missed breaths by evaluating a respiratory exertion flow, an expected respiratory flow, and a determined respiratory flow.

In some embodiments of the system, the detection unit detects the short trigger delays by evaluating a respiratory exertion flow, an expected respiratory flow, and a determined respiratory flow.

In some embodiments of the system, the detection unit detects incorrect triggers and assesses them as asynchrony.

In some embodiments of the system, the calculation unit determines the respiratory exertion flow from the expected respiratory flow and the determined respiratory flow.

In some embodiments of the system, the calculation unit determines the expected respiratory flow from the airway resistance R and the lung elasticity E.

In some embodiments of the system, the calculation unit determines the expected respiratory flow from a mean value of the airway resistance R and a mean value of the lung elasticity E.

In some embodiments of the system, the calculation unit calculates the airway resistance R and the lung elasticity E from the measured values measured by the sensor unit and prepared by the preparation unit.

In some embodiments of the system, the control unit automatically sets the trigger sensitivity of the ventilator on the basis of the missed breaths and short trigger delays detected by the detection unit.

In some embodiments of the system, the control unit automatically sets the trigger sensitivity of the ventilator on the basis of the missed breaths, short trigger delays, and incorrect triggers detected by the detection unit.

In some embodiments of the system, the airway resistance R and the lung elasticity E are determined by the calculation unit via a mathematical lung model.

In some embodiments of the system, the airway resistance R and the lung elasticity E are determined by the calculation unit via a multiple linear regression and the one-compartment lung model.

In some embodiments of the system, the detection unit detects the missed breaths on the basis of at least one of the following features of the respiratory exertion flow, the expected respiratory flow, and the determined respiratory flow:
The local maximum of the determined respiratory flow is between two minima of the respiratory exertion flow
Difference between the temporal position of the local maximum of the respiratory exertion flow and the corresponding left minimum
Difference between the temporal position of the local maximum of the respiratory exertion flow and the corresponding right minimum
Difference between the values of the local maximum of the respiratory exertion flow and the corresponding left minimum
Difference between the values of the local maximum of the respiratory exertion flow and the corresponding right minimum
Expected respiratory flow at the point in time of the local maximum of the respiratory exertion flow
Time between the local maximum of the respiratory exertion flow and the expected trigger point in time.

In some embodiments of the system, the detection unit detects the missed breaths on the basis of the following features of the respiratory exertion flow, the expected respiratory flow, and the determined respiratory flow:
The local maximum of the determined respiratory flow has to be between two minima of the respiratory exertion flow
Difference between the temporal position of the local maximum of the respiratory exertion flow and the corresponding left minimum
Difference between the temporal position of the local maximum of the respiratory exertion flow and the corresponding right minimum
Difference between the values of the local maximum of the respiratory exertion flow and the corresponding left minimum
Difference between the values of the local maximum of the respiratory exertion flow and the corresponding right minimum
Expected respiratory flow at the point in time of the local maximum of the respiratory exertion flow
Time between the local maximum of the respiratory exertion flow and the expected trigger point in time.

In some embodiments of the system, the calculation unit calculates the expected triggering point in time via a mean value of the last breath lengths.

In some embodiments of the system, the calculation unit calculates the expected triggering point in time via a mean value of the length of the last spontaneous breath and the last expected triggering time.

In some embodiments of the system, the mean value is a weighted mean value.

In some embodiments of the system, the detection unit detects on the basis of the value of the trigger delay determined by the calculation unit whether it is a short trigger delay.

In some embodiments of the system, the detection unit detects and determines the trigger delay via the offset between the respiratory exertion of the living being and the triggering of the ventilator.

In some embodiments of the system, the detection unit detects short trigger delays in that the trigger delay is assessed as too short for a respiratory exertion by the living being.

In some embodiments of the system, a trigger delay is detected as a short trigger delay if the trigger delay is less than or equal to a threshold value, wherein this threshold value is selected in a range between 0 seconds and 0.5 seconds, preferably between 0 seconds and 0.25 seconds, particularly preferably between 0 seconds and 0.15 seconds.

In some embodiments of the system, the threshold value for detecting a short trigger delay is 0.1 seconds.

In some embodiments of the system, breaths specified by the ventilator are not studied for trigger delays, wherein the breaths specified by the ventilator are not triggered by a respiratory exertion of the living being.

In some embodiments of the system, the control unit automatically adapts the trigger sensitivity in accordance with the number of detected short trigger delays and missed breaths within a time interval.

In some embodiments of the system, the time interval is between 0.5 and 5 minutes, preferably between 1 and 3 minutes.

In some embodiments of the system, with leakage flows above a threshold value between 15 l/minute and 50 l/minute, the trigger sensitivity is set in the form of an average trigger sensitivity, which is determined with incorporation of triggering sensitivities from prior periods of time having leakage flows below the threshold value.

In some embodiments of the system, with leakage flows of greater than 25 l/minute, the trigger sensitivity is set in the form of an average trigger sensitivity, which is determined with incorporation of triggering sensitivities from prior periods of time having leakage flows less than 25 l/minute.

In some embodiments of the system, the trigger sensitivity describes the parameters, according to which the ventilator detects a breath of the living being and triggers an assistance of the respiratory treatment, wherein the parameters comprise at least a threshold value of the respiratory flow.

In some embodiments of the system, the trigger sensitivity is settable manually and automatically.

In some embodiments of the system, the automatic setting of the trigger sensitivity can set lower threshold values of the parameters for triggering than is possible by the manual setting.

In some embodiments of the system, the parameters for setting the trigger sensitivity are summarized in abstract, unitless numeric values, wherein the numeric values extend from 0 to 8 and wherein a low numeric value reflects a high trigger sensitivity and a high numeric value reflects a low trigger sensitivity.

In some embodiments of the system, values of 0 to 3 are available for the automatic setting of the trigger sensitivity and values from 1 to 8 are available for the manual setting.

In some embodiments of the system, the levels of the trigger sensitivity are defined at least on the basis of threshold values of the respiratory flow.

In some embodiments of the system, the ventilator is configured and designed to provide a recommendation for the manual setting of the trigger sensitivity on the basis of the detected missed breaths and/or short trigger delay.

In some embodiments of the system, the ventilator is configured and designed to generate an alarm if a threshold value for missed breaths and/or short trigger delays is exceeded.

In some embodiments of the system, the ventilator is configured and designed to use at least the missed breaths and/or short trigger delays detected by the detection unit in order to detect unfavorable settings of the ventilator.

In some embodiments of the system, the ventilator is configured and designed to use at least the missed breaths and/or short trigger delays detected by the detection unit in order to detect an intrinsic PEEP.

In some embodiments of the system, the respiratory exertion flow is filtered by a low pass filter and the calculation unit calculates the trigger delay using the filtered respiratory exertion flow.

In some embodiments of the system, the ventilator is configured and designed to detect early expiration triggers.

In some embodiments of the system, the ventilator is configured and designed to assess early expiration triggers as asynchrony.

In some embodiments of the system, the ventilator is configured and designed to detect early expiration triggers on the basis of the time curve of the determined respiratory flow and/or the expected respiratory flow and/or the respiratory exertion flow.

In some embodiments of the system, the ventilator is configured and designed to detect early expiration triggers on the basis of the position and the value of the flow rate of a start of a pressure ramp, an end of a pressure ramp, a local maximum, and/or a local minimum of the determined respiratory flow and/or expected respiratory flow.

In some embodiments of the system, the ventilator is configured and designed to detect early expiration triggers on the basis of at least one of the following points:
the determined respiratory flow at the beginning of the pressure ramp is above the value of the determined respiratory flow at the end of the pressure ramp
the determined respiratory flow is above a value of q₁ at the beginning of the pressure ramp
the determined respiratory flow is below a value of q₂ at the end of the pressure ramp
e*determined respiratory flow (at the local maximum)/determined respiratory flow (at the end of the pressure ramp)<expected respiratory flow (maximum)/expected respiratory flow (at the end of the pressure ramp)
f*determined respiratory flow (at the local maximum)>determined respiratory flow (at the end of the pressure ramp)
g*determined respiratory flow (at the local minimum)<determined respiratory flow (at the end of the pressure ramp)+determined respiratory flow (at the local maximum)
(h<determined respiratory flow (at the local maximum)−determined respiratory flow (at the end of the pressure ramp)) OR (h<determined respiratory flow (local maximum)−determined respiratory flow (at the local minimum))
determined respiratory flow (at the local minimum)<q₃;
in this case, the factors are e in a range from 0.5 to 2.0, preferably between 1 and 1.8; fin a range from 0.9 to 2.9, preferably in a range from 1.7 to 2.3; g in a range from 1 to 3, preferably between 1.8 and 2.2; and h in a range from 2 l/minute to 10 l/minute, preferably between 3 l/minute and 7 l/minute. The values of q₁, q₂, and q₃ are, for example, independently of one another in a range from −5 l/min to +5 l/min, preferably between −1 l/min and +1 l/min.

In some embodiments of the system, the ventilator is configured and designed to detect early expiration triggers on the basis of the following points:
the determined respiratory flow is greater than 0 l/min at the beginning of the pressure ramp
the determined respiratory flow is less than 0 l/min at the end of the pressure ramp
e*determined respiratory flow (at the local maximum)/determined respiratory flow (at the end of the pressure ramp)<expected respiratory flow (maximum)/expected respiratory flow (at the end of the pressure ramp)
f*determined respiratory flow (at the local maximum)>determined respiratory flow (at the end of the pressure ramp)
g*determined respiratory flow (at the local minimum)<determined respiratory flow (at the end of the pressure ramp)+determined respiratory flow (at the local maximum)
(h<determined respiratory flow (at the local maximum)−determined respiratory flow (at the end of the pressure ramp)) OR (h<determined respiratory flow (local maximum)−determined respiratory flow (at the local minimum))
determined respiratory flow (at the local minimum)<0;
   in this case, the factors are e in a range from 0.5 to 2.0, preferably between 1 and 1.8; fin a range from 0.9 to 2.9, preferably in a range from 1.7 to 2.3; g in a range from 1 to 3, preferably between 1.8 and 2.2; and h in a range from 2 l/minute to 10 l/minute, preferably between 3 l/minute and 7 l/minute.

In some embodiments of the system, the trigger sensitivity comprises a value which controls switching from an inspiration phase to an expiration phase, wherein the ventilator is configured and designed to set the value of the trigger sensitivity which controls the switching from an inspiration phase to an expiration phase on the basis of detected early expiration triggers.

In some embodiments of the system, it can be provided that the detection of asynchrony, for example missed breaths, short trigger delays, and/or early expiration triggers, is additionally refined and/or alternatively carried out by the use of parameters such as (respiratory) volume, pressure, flow, and/or (respiratory) frequency. It can be provided, for example, that features of the time curve of the pressure, flow, the respiratory frequency, and/or the respiratory volume are also incorporated in the detection of the asynchrony.

The invention also relates to a method for detecting asynchrony between a ventilator and a living being, wherein the asynchrony in the form of short trigger delays and missed breaths is detected using the lung elasticity E and the airway resistance R of the living being.

It is to be noted that the features listed individually in the claims can be combined with one another in any technically reasonable manner and disclose further embodiments of the invention. The description in particular additionally characterizes and specifies the invention in conjunction with the figures.

It is furthermore to be noted that a conjunction “and/or” used herein, which is between two features and links them to one another, is always to be interpreted so that in a first embodiment of the subject matter according to the invention only the first feature can be present, in a second embodiment only the second feature can be present, and in a third embodiment both the first and the second feature can be present.

A ventilator is to be understood as any device which assists a user or patient in natural breathing, takes over the respiratory treatment of the user or patient, and/or is used for respiratory therapy and/or influences the breathing of the user or patient in another way. This includes, for example, but not exclusively, CPAP and BiLevel devices (sometimes known as BiPAP), narcosis or anesthesia devices, respiratory therapy devices, (clinical, nonclinical, or emergency) ventilators, high flow therapy devices, and coughing machines. Ventilators can also be understood as diagnostic devices for respiratory treatment. Diagnostic devices can be used in general for acquiring medical parameters of a patient. These also include devices which can acquire and optionally process medical parameters of patients in combination with or exclusively relating to the breathing.

A patient interface, if not expressly described otherwise, can be understood as any part or connected peripheral device of the ventilator which is intended for interaction, in particular for treatment or diagnostic purposes, with a patient. In particular, a patient interface can be understood as a mask of a ventilator or a mask connected to the ventilator. This mask can be a full face mask, thus enclosing nose and mouth, or a nose mask, thus a mask only enclosing the nose. Tracheal tubes and so-called nasal cannulas can also be used as a mask.

The system according to the invention is suitable in particular for use in the field of the treatment and respiratory treatment of patients. The system according to the invention is additionally also suitable for use in other fields in which an assistance of the natural breathing can be desired, for example in the case of divers, mountain climbers, in the protective equipment of emergency personnel of the fire department, etc. Moreover, the system according to the invention and the method according to the invention can also be used in nonhuman living beings. It is therefore to be noted that the described exemplary embodiments are designed for humans and in embodiments for nonhuman living beings, parameters such as flow and volume dimensions and also time spans, for example breath lengths, possibly have to be adapted.

Asynchrony is to be understood as deviations of the specified respiratory characteristics of the ventilator and the natural and/or intentional breathing of the living being. For example, it is to be understood as an asynchrony when the living being displays exertion in inhaling, the ventilator does not detect this exertion and accordingly does not trigger assistance for breathing, in particular for inspiration. An asynchrony is also to be understood to mean that a ventilator detects a breath and accordingly triggers an assistance or respiratory treatment, although the living being has not intended a breath. In some embodiments of the invention, the ventilator is set so that from time to time the ventilator triggers a respiratory treatment without a respiratory exertion of the living being detected. This is the case, for example, if the ventilator has not detected a respiratory exertion of the living being over a period of time. Such mandatory or forced respiratory treatments, in contrast, are not understood as asynchrony. In some embodiments, further deviations, for example a phase shift (patient exhales, but ventilator detects an inhale) or time-offset assistance (ventilator reacts too late to respiratory exertions or misses the transition from the inspiration to the expiration) can also be understood as asynchrony.

In the scope of the invention, it is possible to distinguish between at least three types of breaths: spontaneous breaths, missed breaths, and breaths specified by the ventilator. Spontaneous breaths are to be viewed as those breaths which are detected by the ventilator and are accordingly assisted at least in the inspiratory phase, thus the inhalation, by the ventilator. Missed breaths are those breaths in which the living being does display a respiratory exertion, but this is not detected by the ventilator and therefore inspiratory assistance also does not take place. Specified breaths are breaths which are specified by the ventilator without the living being displaying a respiratory exertion or it being detected by the ventilator. A specified breath takes place, for example, when a period of time since the last detected breath is exceeded without a further breath having taken place.

It is to be noted that trigger delays are determined for each triggering of the inspiratory assistance of the ventilator—both for spontaneous and for specified breaths. A trigger delay alone does not yet represent an asynchrony for the system according to the invention, only those trigger delays which are below a defined threshold value, are thus short trigger delays, are assessed as an asynchrony. Short trigger delays are therefore to be viewed as of interest since they do not occur in spontaneous breaths—those breaths intended by the living being—and can be correlated with (forced) triggerings by the ventilator. A strict distinction is thus to be made between short trigger delays and trigger delays (in general). In preferred embodiments of the system, trigger delays for the triggering of an inspiration by the ventilator are not determined without respiratory exertion of the living being.

Furthermore, it is to be noted that the units used, in particular time units, are synonymous in all known notations. The unit “s” and the unit “sec” thus correspond to seconds and can be used interchangeably in three forms (s, sec, second (s)). This also applies for minutes, which are sometimes abbreviated as “min”. In particular for the use of “min” it is to be noted that the unit “min” is not to be confused with the minimum function min( ). In particular at points at which the function min( ) is meant, this is placed in the list with at least the parentheses, wherein accordingly the attributes/values/actions are listed between the parentheses.

According to the invention, the ventilator is equipped at least with a sensor unit, a preparation unit, a calculation unit, a detection unit, a control unit, and a respiratory gas source, such as a fan/valve unit. The sensor unit, preparation unit, calculation unit, detection unit, and control unit each individually or also jointly have a processor, for example, to be able to execute the following steps, such as calculations, analyses, and/or algorithms.

The sensor unit is designed to measure measured values such as pressure and flow with respect to the living being and the ventilator—such as the gas flow and pressure provided by the respiratory gas source. In some embodiments, it can be provided that the sensor unit is also designed to measure further measured values such as temperature, humidity, gas concentrations, volumes, etc.

The preparation unit is configured, for example, to prepare the measured values of the sensor unit and provide them to the system.

The calculation unit is configured, for example, by means of a processor, to further process the prepared measured values and further data, values, and items of information. In particular, the calculation unit is configured to carry out the following calculation steps.

The detection unit is configured, inter alia, to determine data, values, and items of information of various states (inspiration phase, expiration phase, etc.) of the respiration of the living being on the basis of the calculated data. In particular, the detection unit is configured to detect asynchrony between ventilator and living being in the form of short trigger delays, missed breaths, and in some embodiments also incorrect triggerings.

The control unit is designed to control the ventilator 1 at least partially and at least temporarily automatically at least on the basis of the asynchrony detected by the detection unit.

The detection of asynchrony in the form of missed breaths, short trigger delays, and, in some embodiments, incorrect triggerings is based in the system according to the invention on the effective lung parameters of the living being, for example the effective airway resistance R and the effective lung elasticity E. In general, lung parameters are determined in living beings under narcosis and completely passive respiration. However, since the lung parameters are fitted or ascertained here during the respiratory treatment and an active respiration of the living being, the parameters are designated as “effective” parameters.

These effective parameters can be determined, for example, via a mathematical lung model, such as the one-compartment lung model. In some embodiments of the invention, for example, another mathematical lung model, such as the two-compartment lung model or a nonlinear lung model, can also be used.

The one-compartment lung model describes via

EV+R\dot{V}/=P−P_{b}

the relationship between the effective lung elasticity E, the effective airway resistance R, the tidal volume V, the respiratory flow \dot{V}, and a base pressure P_{b} (for example the positive end-expiratory pressure (PEEP)) and the pressure P provided by the ventilator.

The effective lung elasticity E and the effective airway resistance R can be determined, for example, via a multiple linear regression from the one-compartment lung model. For this purpose, it is assumed that the pressure P, the base pressure P_{b}, the tidal volume V, and the respiratory flow \dot{V} are known for each individual breath. For this purpose

$$A = ( \begin{matrix}{V}_{0} & {\dot{V}}_{0} \\ {V}_{1} & {\dot{V}}_{1} \\ {V}_{2} & {\dot{V}}_{2} \\ ⋮ & ⋮ \\ {V}_{n} & {\dot{V}}_{n}\end{matrix} ) ,$$

is defined, wherein the indices designate measurement points at various points in time. Furthermore,

$$x = ( \begin{matrix}E \\ R\end{matrix} )$$

$$and$$

$$Rhs = ( \begin{matrix}{P}_{0} - {P}_{b} \\ {P}_{1} - {P}_{b} \\ {P}_{2} - {P}_{b} \\ ⋮ \\ {P}_{n} - {P}_{b}\end{matrix} ) ,$$

are defined, so that a one-compartment lung model can be expressed as

Ax=Rhs

To determine the effective lung elasticity E and the effective airway resistance R, the technique of multiple linear regression is applied, which results in a linear equation system of the form

A^{t}Ax=A^{t}Rhs,

A^{t} represents the transposed matrix of A here. The following thus results therefrom

$$( \begin{matrix}{\sum }_{i} {V}_{i}^{2} & {\sum }_{i} {V}_{i} {\dot{V}}_{i} \\ {\sum }_{i} {V}_{i} {\dot{V}}_{i} & {\sum }_{i} {\dot{V}}_{i}^{2}\end{matrix} ) ( \begin{matrix}E \\ R\end{matrix} ) = ( \begin{matrix}{\sum }_{i} {V}_{i} ( {P}_{i} - {P}_{b} ) \\ {\sum }_{i} {\dot{V}}_{i} ( {P}_{i} - {P}_{b} )\end{matrix} ) .$$

This linear equation system can be solved for E and R and results in an approximation of the effective airway resistance R and the effective lung elasticity E for a specific breath. The averaged square of the residuum

$$r = \sqrt{{\frac{1}{n} [ ( P - {P}_{b} ) - A x ]}^{2}}$$

can be used to determine an estimation of the accuracy of the approximation of E and R for a specific breath.

The lung parameters E and R can vary according to various influences, such as the position of the living being, sleeping phase, etc., with time. The determined values for E and R can also have a different accuracy for different breaths. To also incorporate the temporal changes and the varying accuracy, the determined lung parameters are filtered before the detection of the asynchrony. The mean values or averages, for example weighted mean values, can be determined for this purpose via various methods.

The exponentially weighted mean values are determined via

Ēₙ₊₁=λĒₙ+(1−λ)Eₙ₊₁

and

R̅ₙ₊₁=λR̅ₙ+(1−λ)Rₙ₊₁,

wherein the indices designate the number of the breath and λ is the so-called forgetting factor, which is used to weight the prior and the current breath appropriately in the mean value. The forgetting factor λ is selected here, for example, according to the time constant

$$τ = - \frac{Δ t}{ln ( λ )}$$

The time constant τ corresponds, for example, to approximately 110 sec to 180 sec, corresponding to a 2 minute interval, wherein a typical breath duration Δt is assumed to be in the range of 3.2 seconds to 4.0 seconds. A typical breath length can generally be assumed to be in a range from 2.0 seconds to 6.0 seconds, preferably a breath length between 3.0 seconds and 4.0 seconds can be assumed, for example. In some embodiments, instead of the 2 minute interval, another interval length can also be selected. The length of the time interval can also be selected, for example, between 1 minute and 10 minutes. The forgetting factor λ can also be adapted accordingly in some embodiments, typically with a value of 0.01 to 1.00. If one assumes, for example, a breath length between 3.0 seconds and 4.0 seconds and one observes an interval having a length between 100 seconds and 180 seconds, λ can thus be between 0.90 and 1.00, preferably between 0.95 and 0.99. For example, the forgetting factor λ is adapted to the assumed typical breath duration and the observed time interval. The breath duration can also be selected variably for the calculation and can be, for example, in a range from 1.0 seconds to 15.0 seconds, preferably between 2.0 seconds and 8.0 seconds.

In some embodiments of the invention, the mean values of R and E can also be determined by other methods, for example by a linear weighted average and/or a logarithmic mean value and/or a square and/or cubed mean value and/or a Gastwirth-Cohen average and/or a combination of various mean values or methods for determining (weighted) mean values.

The calculation of the effective lung elasticity E and the effective airway resistance R forms the foundation for determining further parameters such as the respiratory exertion flow \dot{V}_{eff} and the expected respiratory flow \dot{V}ₑₓₚ, via which in turn asynchrony such as missed breaths, the trigger delay, and possibly incorrect triggerings are detected by the ventilator.

The analysis of the respiratory flow is a fundamental tool for determining asynchrony between the patient and the ventilator. To detect anomalies in the respiratory flow, it is advantageous to know how the behavior of the respiratory flow is to be expected. Differences between the determined and the expected respiratory flow can indicate asynchrony, for example. For ventilated patients, the respiratory flow is strongly dependent on the pressure which is generated by the ventilator. If one solves the differential equation of the one-compartment lung model

P−P_{b}=ĒVₑₓₚ+R̅\dot{V}ₑₓₚ.

by inputting the pressure P, which is generated by the ventilator, and the previously averaged determined lung parameters Ē and R̅, the expected flow \dot{V}ₑₓₚ can be determined. It is moreover assumed that the expected volume Vₑₓₚ is zero at the beginning of a breath. The determined respiratory flow represents the respiratory flow of the patient which is calculated from the flow measured by the ventilator with subtraction of the leakage (suspected/estimated or measured) and other inaccuracies/influences.

If the determined respiratory flow and the expected respiratory flow \dot{V}ₑₓₚ are known, the deviation, the residuum, can be determined as the respiratory exertion flow \dot{V}_{eff}

\dot{V}_{eff}=\dot{V}−\dot{V}ₑₓₚ

which contains items of information with respect to the (unexpected) respiratory exertions of the living being. The respiratory exertion flow \dot{V}_{eff} and/or the time curve of the respiratory exertion flow \dot{V}_{eff} contain key items of information for the algorithm for detecting the asynchrony.

The system according to the invention is configured to execute the algorithm for detecting asynchrony based on the analysis of the respiratory exertion flow and further signals/data/values.

A respiratory exertion of the patient which is not detected by the ventilator and is therefore not assisted by the ventilator by increasing the pressure and/or flow is defined as a missed breath.

The respiratory (or also determined) respiratory flow is typically characterized in a missed breath by a small positive maximum, which is followed by a small negative minimum.

The detection of the missed breaths is executed, for example, by a machine learning algorithm. For this purpose, initial real data of living beings are manually evaluated with respect to the missed breaths and provided to the machine learning algorithm. The machine learning algorithm can then be applied to unknown data or during the use of the ventilator. For example, the machine learning algorithm, after training by the manually evaluated data, is stored on the ventilator and executed by the detection unit.

It is moreover conceivable that the machine learning algorithm achieves further learning advances on the ventilator and these are transmitted from time to time via an interface, for example (in anonymized form) to a server (or a cloud). The collected data can be used via this server to improve the algorithm, for example by higher accuracy in the detection. It can thus be configured that the ventilator updates the machine learning algorithm from time to time using data from the server or provides further learning data. It is also conceivable that the machine learning algorithm learns further from time to time outside the ventilator, for example, by further manually evaluated data, and the learning advances are transmitted from time to time to the ventilator, so that the machine learning algorithm is further improved.

The features, on the basis of which the machine learning algorithm detects the missed breaths on the basis of the respiratory exertion flow, are, for example:
1. The local maximum of the determined respiratory flow has to lie between two minima of the respiratory exertion flow
2. Difference between the temporal position of the local maximum of the respiratory exertion flow and the corresponding left minimum
3. Difference between the temporal position of the local maximum of the respiratory exertion flow and the corresponding right minimum
4. Difference between the values of the local maximum of the respiratory exertion flow and the corresponding left minimum
5. Difference between the values of the local maximum of the respiratory exertion flow and the corresponding right minimum
6. Expected respiratory flow at the point in time of the local maximum of the respiratory exertion flow
7. Time between the maximum of the respiratory exertion flow and the expected triggering point in time.

If at least one of these features is detected, the algorithm can conclude a missed breath. It is preferably provided that multiple, for example two or four or all, of the mentioned features have to apply so that the algorithm concludes a missed breath.

The expected triggering point in time t̅ is determined by the length of the last spontaneous breath t_{current} triggered by the living being itself and the last expected triggering time t̅_{old}

t̅=(1−γ)t̅_{old}+γt_{current},

wherein γ is a factor for weighting t̅_{old} and t_{current}. The value of γ can be selected here between 0.01 and 1.00, preferably between 0.1 and 0.5. For example, a value for γ of ¼ and/or ⅓ and/or ½ can be considered reasonable. The expected triggering point in time can in some embodiments also be defined, for example, by a fixed value. In some embodiments, the triggering point in time is determined by a sliding average, for example over an interval which corresponds to a period of time between 3 and 100 breaths. Other methods for determining an average or mean value can also be used here.

The ventilator is moreover configured to detect asynchrony on the basis of triggered delays or short trigger delays.

Trigger delays occur at the beginning of the spontaneous respiration or the respiratory exertion and are defined as the time interval between the activation of the respiratory muscles of the patient and the triggering of the respiratory assistance by the ventilator. The trigger delays are typically characterized by a local maximum of the respiratory exertion flow. Alternatively or additionally, the trigger delays are characterized by the distance of the maximum in the respiratory exertion from the triggering point in time of the ventilator.

A trigger delay is determined for all triggered breaths. If the trigger delay is less than or equal to a threshold value (for example 0.1 s), which is generally too short for a spontaneous breath, this trigger delay is designated as a “short trigger delay”. While the trigger delay is determined for all breaths (spontaneous and specified by ventilator), an analysis for short trigger delays only takes place for spontaneous breaths, thus breaths not specified by the ventilator. The determination of the trigger delay is carried out as follows, for example:

First the respiratory exertion flow \dot{V}_{eff} is filtered by a low-pass filter, for example a Butterworth filter (for example first, second, third, fourth, and/or fifth order) and/or a Legendre filter and/or a Tschebyscheff filter and/or a Bessel filter and/or a Cauer filter and/or a Gaussian filter and/or a raised cosine filter and/or a TBT filter, a limiting frequency of 3 Hz, to obtain \dot{V}_{eff}^{lowpass}.

The expected amplitude A for the current breath i is then calculated,

A=2 mean[\dot{V}ₑₓₚ(Pₛₑₜ=IPAP & tᵢ≤t<tᵢ₊₁)],

wherein Pₛₑₜ represents the setpoint value of the pressure (pressure setpoint), tᵢ represents the starting point in time of the breath i, and IPAP represents the inspiratory positive airway pressure (IPAP).

Beginning immediately before the trigger point of the inspiration and going back into the expiration phase, each measurement point k is checked as to whether it is part of the trigger delay. Firstly, for this purpose the number n_{highFlow} of the measurement points k having comparatively high respiratory flows above a threshold value of

\dot{V}(k)≥a₁A,

with a₁ between 0.025 and 0.075, for example between 0.050 and 0.060, are calculated.

Following this, the sequence begins again with the last measurement point which was above the threshold value and goes back further, wherein the number n_{increasingFlow} of the measurement points k is counted, which are characterized in that they have a growing respiratory exertion flow and each measurement point k meets the following conditions:

$${\dot{V}}_{eff}^{lowpass} ( k ) > 0 and$$

$$\{ \{ \frac{d}{dt} {\dot{V}}_{eff}^{lowpass} ( k ) > 0 and$$

$$\frac{d}{dt} {\dot{V}}_{eff}^{lowpass} ( k ) > {a}_{2} max ( \frac{d}{dt} {\dot{V}}_{eff}^{lowpass} ( k : k + {n}_{increasingFlow} ) ) \} or$$

$$max ( \frac{{d}^{2}}{{dt}^{2}} {\dot{V}}_{eff}^{lowpass} ( k : k + {n}_{increasingFlow} ) ) < 0 \} ,$$

wherein a2 is selected between 0.3 and 0.6, for example as 0.4 or 0.5. The total number of the test points which correspond to possible trigger delays is

n=n_{increasingFlow}+n_{highFlow}

Finally, it is checked whether the possible trigger delay is valid. This is the case, for example, if

$$mean ( \frac{d}{dt} \dot{V} ( {k}_{start} : k + n - 1 ) ) > {a}_{3} A and$$

$$\dot{V} ( {k}_{start} + n - 1 ) - \dot{V} ( {k}_{start} ) > {a}_{1} A and$$

$${\dot{V}}_{eff}^{lowpass} ( {k}_{start} + {n}_{increasingFlow} ) - {\dot{V}}_{eff}^{lowpass} ( {k}_{start} ) > 0 ,$$

with a₃ between 0.05/s and 0.50/s, preferably between 0.20/s and 0.30/s and kₛₜₐᵣₜ as the first measurement point of the possible trigger delay. If a possible trigger delay is not valid, n is set to 0 (zero). The trigger delay then results as

t_{triggerDelay}=n 0.01s.

An invalid trigger delay is thus assessed in spontaneous breaths corresponding to a short trigger delay. As described above, a trigger delay t_{triggerDelay} less than or equal to a defined threshold value is assessed as a short trigger delay by the ventilator. A value between 0.01 seconds and 0.5 seconds, preferably between 0.05 seconds and 0.15 seconds can be set, for example, for the threshold value.

In some embodiments, the ventilator is furthermore configured to detect incorrect triggerings on the basis of the following evaluations/calculations:

Incorrect triggerings are to be understood as those triggerings which were detected by the ventilator but were not requested by the patient, the patient thus does not display respiratory exertion. These can be detected by analyzing the data in the period of time around the trigger point in time t_{trig} of the ventilator. The normalized respiratory flow before the triggering

fₚᵣₑ=mean(\dot{V}(t_{trig}=tₚᵣₑ≤t<t_{trig}))/A

is calculated in a period of time which is before t_{trig}, wherein the calculation is made with tₚᵣₑ between 0.08 seconds and 0.2 seconds. The normalized respiratory flow after the triggering

$${f}_{post} = \frac{mean ( \dot{V} ( {t}_{trig} \leq t \leq {t}_{trig} + {t}_{post} ) )}{mean ( {\dot{V}}_{exp} ( {t}_{trig} \leq t \leq {t}_{trig} + {t}_{post} ) )}$$

is calculated from the period of time after t_{trig}, wherein the calculation can be made, for example, with a value for tₚₒₛₜ of between 0.1 seconds and 0.5 seconds. An incorrect triggering of the ventilator is detected, for example, if fₚᵣₑ is less than a value between 0.01 and 0.10, for example is 0.05 and fₚₒₛₜ is less than a value between 0.5 and 0.9, for example is 0.7.

The system is also configured and designed, for example, to use the detected short trigger delays and the missed breaths to automatically set the trigger sensitivity of the ventilator. The automatic trigger setting automatically adapts the sensitivity of the ventilator to the triggering for the inhalation in order to meet the individual requirements of the patient.

In general, the trigger sensitivity is set via a specification of levels. The levels of the trigger sensitivity can be reflected in some embodiments on the basis of respiratory flow values, for example in l/min. Thus, for example, levels from 1 l/min to 15 l/min in steps of 1 l/min are conceivable. In addition, intermediate levels can be introduced, for example in 0.5 l/min steps. Other designations for the levels of the trigger sensitivity, for example using numbers, letters, descriptions, and/or symbols, are also possible. Descriptions could assume the form, for example, “very sensitive, sensitive, less sensitive, not sensitive”. In addition to the designation of the steps (numeric, alphabetic, symbolic, by flow values, etc.), the number and/or the spacing of the available levels can also vary.

In some embodiments of the system, the trigger sensitivity can be set, for example, manually in levels between 1 and 8, wherein, for example, smaller values correspond to a higher sensitivity—for example, related to a lower respiratory flow which triggers the assistance by the ventilator. For the automatic trigger setting, for example, a further level 0 of the trigger sensitivity is added, which corresponds to an even higher sensitivity. This higher trigger sensitivity can be especially useful for COPD patients having an intrinsic positive end expiratory pressure (iPEEP), but also for other clinical pictures or cases in which the system or ventilator according to the invention is used. The automatic trigger setting is designed so that the sensitivity levels between 0 and 3 are set thereby. In some embodiments, a trigger sensitivity between 0 and 8 can also be set by the automatic trigger setting. An excessively high sensitivity (corresponds to small levels of the trigger sensitivity) can be viewed as undesirable or annoying under certain circumstances in areas of use outside respiratory therapy. The levels reflect, inter alia, threshold values for the respiratory flow at which an inspiration or the assistance during the inspiration is triggered.

The adaptation of the trigger sensitivity via the automatic setting (also auto-trigger function) is based on the number of the established missed breaths and short trigger delays in a predetermined time interval (for example 2 minutes, other time intervals can also be used here, which are ideally matched to the time intervals of the calculation or the detection of the asynchrony). The following mutually exclusive rules apply for the adaptation of the trigger sensitivity
1. IF (#ShoTrigDel≥b #IneffEff) & (#ShoTrigDel≥c)→trigSens=min(trigSens+1.3)
2. IF [(#IneffEff≥b #ShoTrigDel) & (#IneffEff≥c)]→trigSens=max(trigSens−1.0)
3. IF (#ShoTrigDel≤d) & (#ShoTrigDel_{old}≤d) & (trigSens=trigSens_{old})→trigSens=max(trigSens−1.1)
   wherein #IneffEff indicates the number of the registered missed breaths in the time interval, #ShoTrigDel stands for the number of the short respiratory trigger delays in the same interval, the index “old” describes the preceding time interval, the parameters b, c, d are, for example, in a range
b=0 to 6,
c=0 to 6,
d=0 to 6
   and trigSens is the current trigger sensitivity level. In particular, the parameters b, c, d can assume values from 0 to 6, for example from 0 to 4 or from 0 to 3. It can be provided here that b=c and c>d applies. The parameter d has a value less than 1, for example. The comma separates in this case the two parameters of the minimum function min( ) and the maximum function max( ). Here, min(i,j) and max(i,j) are functions. In this case, min( ) would return the smaller value of i and j whereas max( ) would have the greater value as the result. Example: min(2,4)=2 and max(2,4)=4. Accordingly, trigSens=min(trigSens+1,4) means that the variable trigSens is increased by 1, but at most can assume the value 4. Example: trigSens has the value 3. After the assignment trigSens=min(trigSens+1,4), trigSens has the new value min(4,4)=4. If trigSens now has the value 4 and the assignment trigSens=min(trigSens+1,4) is executed, trigSens has the new value min(5,4)=4. TrigSens thus cannot become greater than 4 in this way.

Instead of numeric values for trigSens, as described above, other expressions are also possible, for example words, letters, and/or symbols. If numeric values are not used for trigSens, the adaptation sequence accordingly has to be adapted so that the respective step adaptation can take place. It can additionally or alternatively also be provided that the display of the trigger sensitivity is carried out using letters, symbols, descriptions, and/or flow values, wherein the corresponding numeric scale is associated with the respective displayed type.

In some embodiments, b, c, d can also assume other values, for example between 1 and 6, preferably between 2 and 4. The value ranges specified here for b, c, d can in some embodiments also be mixed with those mentioned above. For example, a value range of 1 to 6 can underlie b, c, while a value range from 0 to 6 applies for d. Furthermore, it is conceivable, for example, that the entire preceding interval is described by the 2 minute interval and the next interval begins after the prior interval has been ended. The first interval thus begins at second 0 and lasts until second 120. The second interval follows seamlessly and lasts from second 120 to second 240. The selected interval length can additionally be in a range between 60 seconds and 240 seconds, preferably between 100 seconds and 180 seconds.

The number of the missed breaths and the number of the short trigger delays become more inaccurate for cases having high leakage flows. Therefore, if the average leakage flows in the 2 minute interval are above a value between 15 l/min and 501/min, for example 25 l/min, the trigger sensitivity is set as an average trigger sensitivity

T̅_{new}=(1−λ_{Leak})T̅_{old}+λ_{Leak}T

which is calculated from the prior periods of time having lower or no leakage flows. In this case, T̅_{new} and T̅_{old} are the new (index new) and the old (index old) average values, T is the trigger sensitivity calculated for the current period of time, and λ_{Leak} is the associated forgetting factor, which can be assigned, for example, a value between 0.01 and 1.00. The value is preferably between 0.05 and 0.40 and/or between 0.15 and 0.30. The forgetting factor is a factor which is calculated in, in order to weight the preceding and the current period of time according to the 2 minute interval or which defines the timescale of the filtering—approximately two minutes here. In some embodiments, for example, it is determined in dependence on the leakage flow whether and/or to what extent adaptations of the trigger sensitivity T̅ are performed. For example, if a certain leakage flow is exceeded, the adaptation of the trigger sensitivity T̅ is omitted. The threshold value of the leakage flows above which the adaptation is omitted is, for example, in the range between 15 l/min and 50 l/min, preferably between 20 l/min and 30 l/min. The threshold value can also relate to an average value of the leakage flows, for example, the adaptation of the trigger sensitivity T̅ can also be omitted from and/or above a threshold value of average leakage flows between 15 l/min and 50 l/min, preferably between 20 l/min and 30 l/min. The trigger sensitivity T̅ is not adapted, for example, for phases having high average leakage flows above 25 l/min.

In some embodiments, the system is also configured to detect early expiration triggers, so-called “early cyclings” and assess them as asynchrony. Early expiration triggers (early cyclings) occur when the ventilator switches from the inspiration to the expiration although the user/the patient has not yet completed the inspiration phase. The patient is caused to exhale due to the pressure drop, which is typically followed by a short increase of the respiratory flow, since the patient has not yet completed his inspiration or wishes to inhale further. The patient often ultimately adapts himself to the ventilator and begins with expiration.

Four fundamental points in the respiratory flow curve are to be considered in the determination of the early expiration triggers: the start of the pressure ramp from inspiration to expiration (by the ventilator), the end point of the pressure ramp, the position of the local maximum of the respiratory flow, which is not to be located farther than a certain time span after the end point of the pressure ramp, and the local minimum, which is to follow within a specific time span after the end point of the pressure ramp. The time span for the local maximum after the end point of the pressure ramp is, for example, between 0.1 seconds and 2 seconds, for example between 0.3 and 0.4 seconds. The time span for the local minimum after the end point of the pressure ramp is, for example, between 0.2 seconds and 3 seconds, for example between 0.5 seconds and 0.7 seconds. The early expiration triggers are to be observed at the beginning of the expiration phases and are characterized by a local maximum of the determined respiratory flow which are not to be observed in the expected respiratory flow. Accordingly, the respiratory exertion flow reflects a local maximum after the time of the early expiration trigger. Instead of a pressure ramp, alternatively or additionally a flow ramp can also be used or observed.

An early expiration trigger is detected, for example, if at least one of the following conditions applies:
1. Determined respiratory flow (beginning of the pressure ramp)>0
2. Determined respiratory flow (end of the pressure ramp)<0
3. e*determined respiratory flow (local maximum)/determined respiratory flow (end of the pressure ramp)<expected respiratory flow (maximum)/expected respiratory flow (end of the pressure ramp)
4. f*determined respiratory flow (local maximum)>determined respiratory flow (end of the pressure ramp)
5. g*determined respiratory flow (local minimum)<determined respiratory flow (end of the pressure ramp)+determined respiratory flow (local maximum)
6. (h<determined respiratory flow (local maximum)−determined respiratory flow (end of the pressure ramp)) OR (h<determined respiratory flow (local maximum)−determined respiratory flow (local minimum))
7. Determined respiratory flow (local minimum)<0

In this case, the factors are e in a range from 0.5 to 2.0, preferably between 1 and 1.8; fin a range from 0.9 to 2.9, preferably in a range from 1.7 to 2.3; g in a range from 1 to 3, preferably between 1.8 and 2.2; and h in a range from 2 l/min to 101/min, preferably between 3 l/min and 7 l/min.

In some embodiments, to detect early expiration triggers, it can also be provided that instead of conditions 1 and 2, it is generally to apply that the determined respiratory flow at the beginning of the pressure ramp is greater than the determined respiratory flow at the end of the pressure ramp. In some embodiments, the threshold values for conditions 1, 2, and 7 can also independently of one another be above and/or below 0 (l/min).

In some embodiments, it is provided that at least 2 or 4 or more or all of the mentioned conditions have to apply to detect early expiration triggers.

It is to be noted that a joint detection of the asynchrony from the respiratory flows or the respiratory exertion flow is possible. The described possibilities for detection, which are described individually by way of example, can be combined accordingly. The following descriptions on the basis of the figures also partially each represent the detection of individual asynchrony. A combination of these detections, for example, to simultaneously detect missed breaths and/or short trigger delays and/or incorrect triggerings and/or early expiration triggers, can also be possible. Accordingly, the trigger sensitivity can also be controlled at least partially on the basis of the jointly detected asynchrony.

In some embodiments, the trigger sensitivity comprises at least one value for switching the ventilator into the inspiration phase, in which the inhalation of the user is assisted, and optionally at least one value for switching into an expiration phase, which assists the user in exhalation. The expiration phase differs from the inspiration phase, for example, in that a lower pressure and/or lower flow is specified by the ventilator.

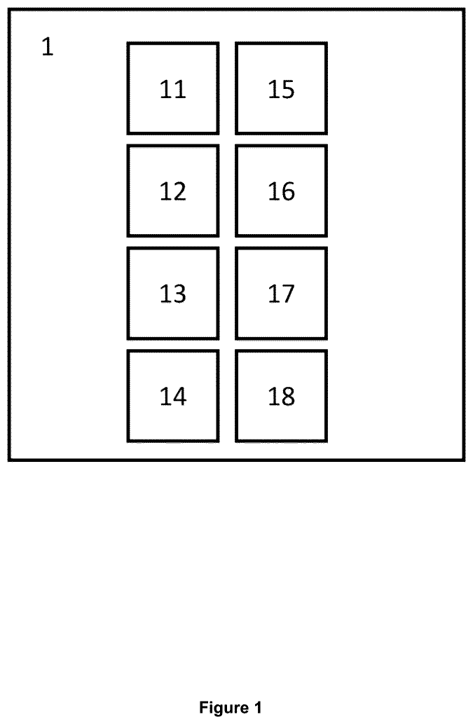

The invention is explained in more detail hereinafter on the basis of FIGS. 1 to 7 via exemplary embodiments.

FIG. 1 shows by way of example a ventilator 1 having a sensor unit 11, a preparation unit 12, a calculation unit 13, a detection unit 14, a storage unit 15, a monitoring unit 16, a control unit 17, and a fan/valve unit 18. The units 11, 12, 13, 14, 16, 17 can, for example, be part of a computer program, which is executed by a processor on the ventilator 1. A combination of the units 11, 12, 13, 14, 15, 16, 17 in a control unit would also be conceivable, for example.

The sensor unit 11 is configured to acquire measured values, in particular parameters, which are related to a respiratory flow, a respiratory volume, a respiratory frequency, an inhalation and exhalation duration, a respiratory contour, a leakage, or a treatment pressure. The sensor unit 11 can optionally perform additional measurements of components or temperature of the respiratory gas or the blood. The sensor unit 11 transmits the acquired measured values to the preparation unit 12.

The preparation unit 12 can prepare the acquired measured values. For example, the preparation unit 12 can carry out smoothing, artifact filtering, or downsampling of the measured values.

The calculation unit 13 calculates, from the measured values acquired by the sensor unit 11 and prepared by the preparation unit 12, signals and/or key values, for example a mean value, a median, a percentage, a derivative, a frequency distribution, a duration, or a proportion of exceeding or falling below threshold values.

The detection unit 14 is configured to detect events/states such as alarms, breathing stops, artifacts, coughs, oxygen (de-)saturations, asynchrony 2 between device and user, missed breaths 218, trigger delays 305, incorrect triggerings 307, inhalations, exhalations, and/or mandatory breaths.

The storage unit 15 stores, inter alia, the values/parameters acquired by the sensor unit 11 and/or the values, data, and/or items of information prepared by the preparation unit 12 and/or the calculation unit 13, for example at least stores them temporarily. The items of information, data, and values obtained by the detection unit 14 also can be and/or are at least temporarily stored in the storage unit. Temporary storage means, for example, that the values, data, and/or items of information are stored until a transmission and are then erased or are authorized to be overwritten, for example.

The monitoring unit 16 acquires, for example, technical problems of the ventilator 1. Technical problems 1 can be, for example, a low battery level, faults in the electronics, a defective battery, a defective component, a power failure, an incorrectly functioning accessory part, an implausible measured value, or leaving a permitted temperature range. The monitoring unit 17 can display or transmit an alarm on the ventilator 1 via an interface in the event of a detected technical problem.

The control unit 17 is used, for example, to control the ventilator 1, in particular a fan and/or valve unit 18 for generating the respiratory gas flow or ventilation pressure. The control unit 17 can also be designed to control other components and/or units of the ventilator 1. In some embodiments, the control unit 17 can also be subdivided further and can consist of multiple control units, which each control an individual unit and/or component of the ventilator 1. In particular, the control unit 17 is configured to control the ventilator 1 at least partially automatically on the basis of the ascertained data, values, and findings of the sensor unit 11, the preparation unit 12, the calculation unit 13, and/or the detection unit 14. In some embodiments, the control unit 17 is configured so that the control takes place partially on the basis of manually set parameters and partially on the basis of automatically set parameters. In some embodiments, the control can also take place exclusively on the basis of manual settings or exclusively automatically.

The ventilator 1 is configured, for example, for the purpose of specifying a uniform respiratory gas pressure (for example in the form of a CPAP therapy) and/or of switching the specified respiratory gas pressure between the expiration phases and inspiration phases (for example in the form of a bilevel ventilator). For example, a higher respiratory gas pressure is specified during the inspiration phase than in the expiration phase. In some embodiments, a switch between inspiration and expiration and/or between expiration and inspiration takes place in the form of a pressure ramp, so that the pressure and/or flow specification is not abruptly changed. The switch can also take place in the form of a flow ramp instead of via a pressure ramp. The point at which a switch is made between inspiration and expiration or expiration and inspiration can be at least partially determined, for example, via a trigger sensitivity.

The detection unit 14 is configured, for example, for the purpose of detecting asynchrony 2 between the ventilator 1 and the connected living being. An asynchrony 2 is present, for example, if the living being wishes to inhale, but the ventilator 1 does not detect this, for example, and does not trigger any assistance during the inspiration phase. The reverse case, the living being does not wish to inhale, but the ventilator 1 incorrectly detects an exertion of the living being to inhale and accordingly triggers the inspiration assistance, can also count as an asynchrony 2. A planned, forced inhalation by triggering the inspiration assistance by the ventilator 1, for example, because the living being exceeds a certain time since the last inspiration or the maximum specified time of the expiration was exceeded, is not assessed as an asynchrony 2 in most embodiments. The data and values which are measured and/or ascertained during a forced inspiration are therefore also generally not used to assess the asynchrony 2.

The detection of the asynchrony 2 is carried out, for example, during the use of the ventilator 1 by the living being. Accordingly, the results of the detection are immediately generated “live” and possibly also used directly by the ventilator 1, for example for the control.

The exemplary embodiment of the system in FIGS. 1 to 3 detects missed breaths 218, thus events in which the living being wishes to inhale, but the ventilator 1 has not triggered assistance in the inspiration, and short trigger delays 308, thus events in which the living being did not wish to inhale, but the ventilator 1 has triggered an inspiration assistance, as asynchrony 2. The trigger delays 305 which are determined to be equal or less than, for example, 0.1 seconds, are designated here as short trigger delays 308. In these short trigger delays 308, it is presumed that the living being did not wish to inhale. In some embodiments, the system is also configured so that asynchrony 2 in the form of incorrect triggerings 307 is detected. While the short trigger delays 308 can be viewed as an indication of a trigger without the intention of inhaling by the living being, the system detects these events with greater certainty in the case of incorrect triggerings 307.

The detection unit 14 detects the short trigger delays 308 and the missed breaths 218 from the respiratory exertion flow, the expected respiratory flow, and the determined respiratory flow.

The determined respiratory flow represents the respiratory flow of the patient which is calculated from the flow measured by the ventilator with subtraction of the leakage (suspected/estimated or measured) and other inaccuracies/influences. The calculations which form the basis of the detection of the asynchrony 2 by the detection unit 14 are carried out, for example, by the calculation unit 13. The expected respiratory flow is determined from the effective airway resistance R and the effective lung elasticity E. These are in turn calculated via a mathematical lung model, for example the one-compartment lung model. The respective parameters R and E can be determined, for example, via multiple linear regression from the lung model used.

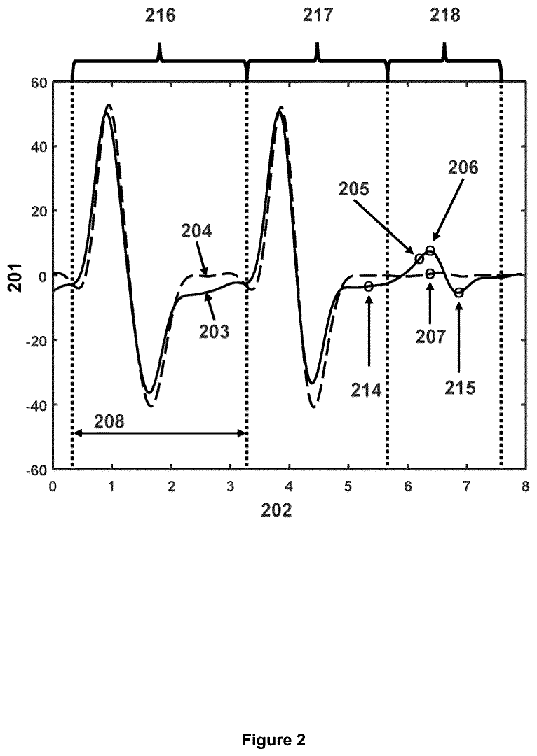

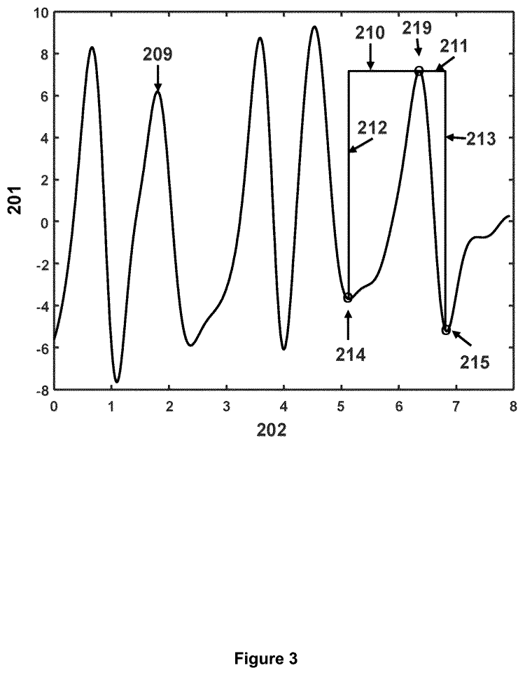

The detection of the asynchrony 2 in the form of missed breaths 218 is schematically shown in FIGS. 2 and 3. The time curve of the determined respiratory flow 203 and the expected respiratory flow 204 is schematically plotted in FIG. 2 in a diagram with the time 202 as the x axis and the flow rate 201 on the y axis. The duration of a breath corresponds to the time interval 208, wherein a breath can be roughly divided into the inspiration and expiration. The inspiration can essentially be detected by a positive respiratory flow. The expiration is essentially characterized by a negative respiratory flow, wherein the respiratory flow at the end of the expiration becomes less—thus in the direction of positive values, but nonetheless negative—and finally approaches the zero value with a flatter slope. Two complete breaths 216, 217 are shown in the diagram of FIG. 2b, detectable, for example, at the positive and negative peaks of both the determined respiratory flow 203 and the expected respiratory flow. Moreover, a missed breath 218 is also shown by way of example. The missed breath 218 can be detected, for example, in that both positive and negative peak of the determined respiratory flow 203 have significantly smaller values than those of the complete breaths 216, 217. An assistance by the ventilator 1 was not triggered, which can be seen, for example, from the expected respiratory flow 204 in FIG. 2.

The detection unit 14 detects missed breaths 218 reliably on the basis of the respiratory exertion flow 209, which is plotted in the diagram in FIG. 3. Alternatively or additionally, it can be provided that the missed breaths 218 can also be detected on the basis of features of the respiratory flow and/or the pressure and/or the respiratory frequency and/or the respiratory volume. In the diagram, the flow rate 201 (y axis) of the respiratory exertion flow is plotted against the time 202 (x axis). The detection unit 14 checks on the basis of the values of the respiratory exertion flow 209, the time curve of the respiratory exertion flow 209, the determined respiratory flow 203, the expected respiratory flow 204, and the expected trigger points in time, for example, the following features to detect a missed breath 218:
1. The local maximum 206 of the determined respiratory flow 204 has to lie between two minima 214, 215 of the respiratory exertion flow 209.
2. Difference 210 of the temporal position of the local maximum 219 of the respiratory exertion flow and the corresponding left minimum 214.
3. Difference 211 of the temporal position of the local maximum 219 of the respiratory exertion flow and the corresponding right minimum 215.
4. Difference 212 between the values of the local minimum 219 of the respiratory exertion flow and the corresponding left minimum 214.
5. Difference 213 between the values of the local maximum 219 of the respiratory exertion flow and the corresponding right minimum 215.
6. Expected respiratory flow 207 at the point in time of the local maximum 219 of the respiratory exertion flow 209.
7. Time between the local maximum 219 of the respiratory exertion flow and the expected trigger point in time 205.

The detection unit 14 is configured, for example, so that these features are checked on the basis of a machine learning algorithm. For example, for this purpose multiple data of living beings are manually evaluated and provided to the machine learning algorithm, which derives values, data, parameters, and items of information therefrom, using which the missed breaths 218 are detected. For example, a machine learning algorithm based on the AdaBoost M1 technology can be used for this purpose.

The detection unit 14 thus detects, for example, on the basis of at least one of the features that a missed breath is present. In some embodiments, it is provided that multiple, for example at least two, four, or all of the features have to be used in the detection of the missed breaths or the criteria have to be met so that the respective feature indicates a missed breath.

In some embodiments, it can be provided that the detection of missed breaths is carried out and/or further refined alternatively or additionally by the use of further parameters such as pressures, flows, volumes, frequencies.

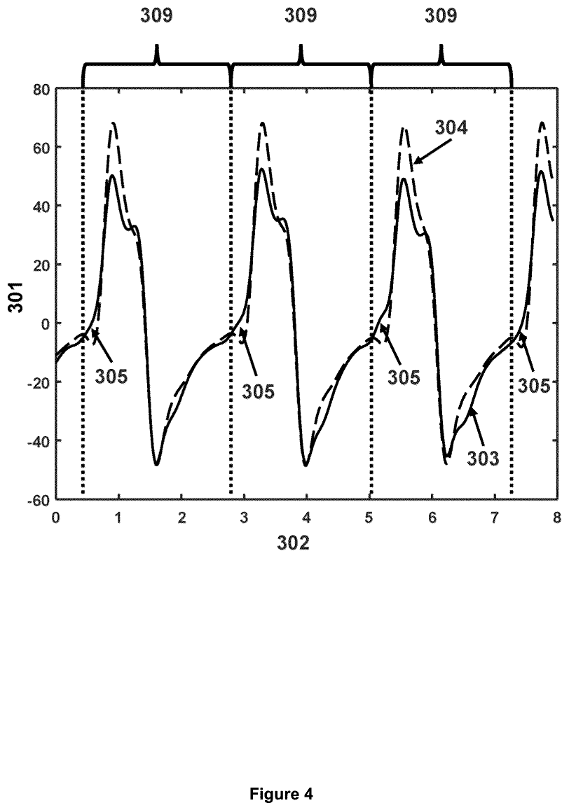

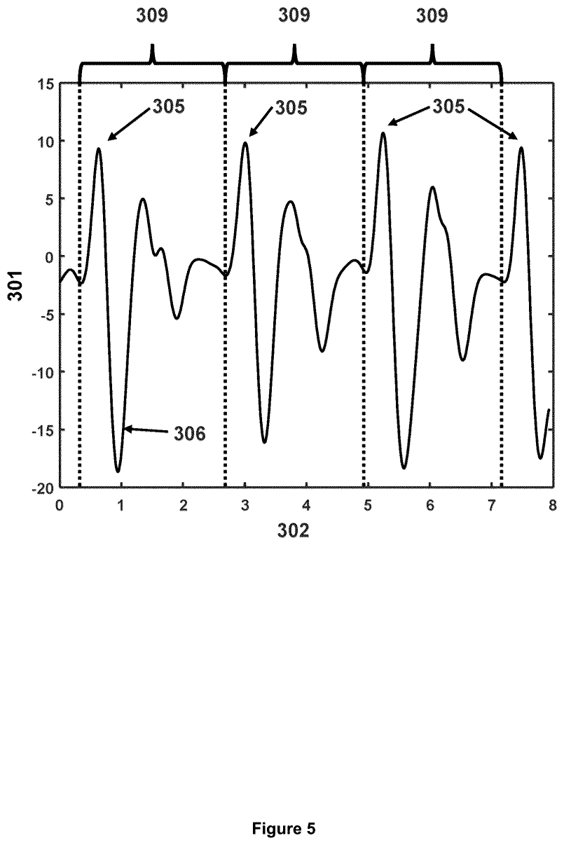

Furthermore, the detection unit 14 is also configured, for example, to detect or determine trigger delays 305 and to assess short trigger delays 308 as an asynchrony 2 between the ventilator 1 and the living being. Trigger delays 305 are determined, for example, for all breaths with inspiratory assistance, a check for short trigger delays 308 is only carried out, for example, for spontaneous breaths. Trigger delays 305 are shown by way of example in FIGS. 4 and 5. FIG. 4 shows a diagram in which the flow rates 301 of the determined respiratory flow 303 and the expected respiratory flow 304 are plotted against the time 302. The time interval between the activation of the respiratory muscles of the living being and the triggering of the respiratory assistance, for example for inspiration, by the ventilator 1 is defined as a trigger delay 305. In FIG. 4, the trigger delays 305 can be detected, for example, from the offset between the beginning of the slope of the flow rate of the determined respiratory flow 303 and the expected flow rate 304. If one plots the flow rate 301 of the respiratory exertion flow 305 against the time 302, as can be seen in the diagram in FIG. 5, the trigger delays 305 can thus be detected at the local maximum of the breaths 309.

Trigger delays 305, the value of which does not exceed a threshold value of, for example, 0.1 seconds, are detected as short trigger delays 308. In some embodiments, this threshold value can also be selected to be greater, for example up to 0.5 seconds, or less, for example 0.05 seconds.

The trigger delays 305 are determined, for example, by computer and via an algorithm using the respiratory exertion flow 305. For this purpose, a low-pass filter, here, for example, a third-order Butterworth filter, having a limiting frequency of 3 Hz is applied to the respiratory exertion flow 306. Moreover, an expected amplitude A for the current breath i is calculated using the respiratory exertion flow 306. For each measurement point k, beginning immediately before the trigger point of the inspiration and going back into the expiration phase, it is checked whether this measurement point k is part of the trigger delay—thus lies between the beginning of the respiratory exertion of the living being and the triggering of the ventilator 1. A measurement point k corresponds, for example, to the measured values recorded by the sensor unit 11 at a point in time, which are possibly further processed by the preparation unit 12 and the calculation unit 13. The measurement points k are checked with respect to the respiratory flow as to whether they reach or exceed a specific threshold value, for example a₁ times the expected amplitude A, and are counted as the number n_{highFlow}. The factor a₁ can assume values between 0.005 and 0.1, for example, preferably between 0.025 and 0.075. In some embodiments, the factor a₁ is defined with a value between 0.05 and 0.06.

Starting from the last measurement point which was above the threshold value, looking backwards in time, the number n_{increasingFlow} of the measurement points is counted which are characterized by a rising respiratory exertion flow 306 and meet further conditions. The total number of the measurement points of a possible trigger delay 305 corresponds to the sum n of n_{highFlow} and n_{increaisngFlow}. Furthermore, it is finally checked on the basis of the measurement points k whether the calculation of the trigger delay 305 is valid. If the calculation of the trigger delay 305 should not be valid, the sum n is thus set as 0. The length t_{triggerDelay} of the trigger delay 305 results from the multiplication of the sum n with a factor t_{tD}. The factor t_{tD} is, for example, between 0.001 seconds and 0.05 seconds, preferably between 0.005 seconds and 0.015 seconds. If the value of t_{triggerDelay} is, for example, at 0.1 seconds or less, the ventilator 1 thus detects a short trigger delay 308 by way of the detection unit 14, which is assessed as an asynchrony 2.

In some exemplary embodiments, the ventilator 1 having the detection unit 14 is configured so that incorrect triggerings 307 can be identified and these can be detected as an asynchrony 2. For this purpose, the data and measured values around the trigger point in time t_{trig} of the ventilator 1 are analyzed. For this purpose, the normalized respiratory flows before (fₚᵣₑ) and after (fₚₒₛₜ) the triggering are calculated. If the values for fₚᵣₑ and fₚₒₛₜ exceed specific individual threshold values, a correct triggering is detected. If fₚᵣₑ and fₚₒₛₜ fall below these values or reach them, an incorrect triggering 307 is detected. The threshold value of fₚᵣₑ is defined, for example, in a range from 0.005 to 0.5, preferably between 0.025 and 0.075. The threshold value for fₚₒₛₜ is defined, for example, at a value between 0.1 and 1.5, preferably between 0.5 and 1.0. For example, an incorrect triggering 307 is detected if fₚᵣₑ≤0.075 and fₚₒₛₜ≤0.8 apply.

The ventilator 1 can automatically set the trigger sensitivity 3 on the basis of the detected asynchrony 2. In some embodiments, an option for the automatic setting of the trigger sensitivity 3 can be selected into various manual levels for the trigger sensitivity 3. For example, a level from 1 to 8 or the automatic trigger sensitivity 4 can be selected on the ventilator 1 for the trigger sensitivity 3. The trigger sensitivity 3 reflects, for example, in an abstract or unitless number the sensitivity with which the ventilator 1 triggers the inspiration assistance. For example, at least a threshold value for the respiratory flow at which the inspiration assistance by the ventilator 1 is triggered, is taken into consideration by the trigger sensitivity 3. In some exemplary embodiments, the trigger sensitivity 3 is primarily and/or only dependent on a threshold value of the respiratory flow. A higher level of the trigger sensitivity 3 means, for example, among other things, a higher threshold value for the respiratory flow at which the inspiration assistance is triggered. For example, level 1 thus represents a more sensitive trigger sensitivity 3 than level 2.

The trigger sensitivity 3 can be set or selected, for example, via a user interface—for example a display device embodied as a touchscreen and/or input devices on the ventilator 1. A setting/selection of the trigger sensitivity 3 via a remote station separate from the ventilator 1 can also be possible.

If the automatic setting of the trigger sensitivity 3 is selected, the ventilator 1 thus sets the trigger sensitivity 3 automatically and takes into consideration at least the detected asynchrony 2, for example missed breaths 218 and short trigger delays 308. In some embodiments, the ventilator 1 additionally also takes into consideration detected incorrect triggerings 307 in the automatic setting of the trigger sensitivity 3. The ventilator 1 can automatically set the trigger sensitivity 3, for example, to levels 0 to 3. Level 0 represents an even more sensitive trigger sensitivity 3 than level 1. Level 0 is only available for the automatic setting, for example, thus cannot be set manually. However, it is also conceivable that all steps are available for both the manual and the automatic setting of the trigger sensitivity 3—thus levels from 0 to 8 can be set both manually and automatically. In addition to a division into levels from 0 to 8, the trigger sensitivity 3 can also be divided into another arbitrary number of levels. These can be characterized, for example, by numbers, letters, or also by descriptions, for example “very sensitive, sensitive, less sensitive, not sensitive”. In some embodiments, the levels of the trigger sensitivity 3 are specified by the flow values, for example in l/min. The trigger sensitivity 3 can thus be settable, for example, between 1 l/min to 25 l/min or 1 l/min to 10 l/min.

The trigger sensitivity 3 is reflected, for example, via the parameter trigSens. In the case of a manual setting of the trigger sensitivity 3, this parameter is changed for the setting, for example, directly by an input via an interface (not shown in FIG. 1), for example a user interface. The parameter trigSens is determined in the automatic setting depending on the number of the short trigger delays 308 and missed breaths 218 in the current and last time interval. The time interval is a 2 minute interval in the exemplary embodiment shown. This time interval is matched, for example, to the preceding time intervals for determining and/or detecting the asynchrony 2. The values of the parameter trigSens correspond here to the levels of the trigger sensitivity 3.

The trigger sensitivity 3 is set via the minimum function min(i,j) and/or the maximum function max(i,j). The min( ) function supplies the smaller value of i and j again here—min(3,4) would thus be 3. The max( ) function again supplies the greater value of i and j—max(1,4) would accordingly be 4.

A set of rules is established, according to which the trigger sensitivity 3 is adapted. For example, three rules follow, which mutually exclude one another:
1. IF (#ShoTrigDel≥b #IneffEff) & (#ShoTrigDel≥c)→trigSens=min(trigSens+1.3)
2. IF [(#IneffEff≥b #ShoTrigDel) & (#IneffEff≥c)]→trigSens=max(trigSens−1.0)
3. IF (#ShoTrigDel≤d) & (#ShoTrigDel_{old}≤d) & (trigSens=trigSens_{old})→trigSens=max(trigSens−1.1)
   #ShortTrigDel corresponds here to the number of registered short trigger delays 308 during the current time interval, #IneffEff corresponds to the number of the registered missed breaths 218, and trigSens represents the current value or the level of the trigger sensitivity 3. The index old furthermore indicates the values of the preceding time interval. In this case, b, c, and d are parameters in a range from 0 to 6, for example in a range from 0 to 3. In some embodiments, the parameters b, c, d can be assigned, for example, values from 0 to 2, wherein b and c can have the same value and d has a lower value, for example less than 1.

In some embodiments, b, c, and d can also assume other values, it is to be noted in this case, however, that the three above-mentioned rules still mutually exclude one another, thus only one rule at a time is met or active. In some embodiments, the third rule can also be modified so that the trigger sensitivity 3 (trigSens) is not changed if no asynchrony 2 is registered, independently of which level is currently set for the trigger sensitivity 3.

The value trigSens is changed according to the minimum function min( ) or the maximum function max( ). Due to the use of the minimum and maximum function, it is thus implemented that certain values—depending on fulfilling the rules—cannot be exceeded and/or fallen below. In this example, the smallest possible level is already set with 0. If a more sensitive level below 0, for example −1, is defined for the trigger sensitivity in another embodiment of the ventilator 1, this can be made accessible for the automatic setting via the second rule.

In addition to the rules shown, it is moreover also possible that further rules are defined, on the basis of which the ventilator 1 can automatically set the trigger sensitivity 3. It is also to be noted here that the defined rules mutually exclude one another. In some embodiments, the ventilator 1 or the detection unit 14 is configured to detect incorrect triggerings 307. Accordingly, the existing rules can be expanded with the detected incorrect triggerings 307.

Additional rules can also be defined, for example, which take into consideration the incorrect triggerings 307. These can also be worded, for example, so that following one of the first three rules does not exclude following at least one of the rules which take into consideration the incorrect triggerings 307. In addition to one of the first three rules, at least one of the further rules can thus also be followed.

In some embodiments, it is presumed, for example, that the detection of asynchrony 2 becomes more inaccurate when the leakage flows rise, for example, above a value between 15 l/min and 50 l/min, for example 25 l/min. If leakage flows of 25 l/min or higher are detected by the ventilator 1 for a time interval, for example a 2 minute interval, the ventilator 1 is thus configured to set an average trigger sensitivity T̅_{new} for the next and/or current time interval. This average trigger sensitivity T̅_{new} is calculated from the average trigger sensitivity T̅_{old} of the last time interval and the calculated current trigger sensitivity T:

T̅_{new}=(1−λ_{Leak})T̅_{old}+λ_{Leak}T

The factor λₗₑₐₖ designates here a forgetting factor which weights the value of the old average trigger sensitivity T̅_{old} in relation to the current trigger sensitivity T. For example, λₗₑₐₖ is assigned a value between 0.01 and 0.9, preferably between 0.1 and 0.5. For example, λₗₑₐₖ is assigned the value 0.2 in the exemplary embodiment. In some embodiments, the average trigger sensitivity within lasting phases having high leakage flows above a threshold value of, for example, 25 l/min is not further adapted. That is to say that an average trigger sensitivity is recalculated once after a first time interval, for example 2 minutes, of high leakage flows over 25 l/min and is then maintained as long as no time interval having leakage flows below 25 l/min is registered.

In some embodiments of the system, during the usage time, with set manual trigger sensitivity 3, the asynchrony detection is carried out and after ending the use a summary is prepared of the detected asynchrony 2. This summary can, for example, moreover also contain recommendations for the manual setting of the trigger sensitivity 3. These recommendations can be output or displayed, for example, on a display of the ventilator 1 or via an interface for telemonitoring.

Moreover, the generation of an alarm by the ventilator 1 is also conceivable, if a threshold value of detected missed breaths 218, short trigger delays 308, and/or incorrect triggerings 307 should be exceeded. For example, this threshold value can be a percentage proportion of the total detected breaths per time interval. The time interval can certainly be selected to be greater here than the time intervals used for asynchrony detection. The percentage proportion of detected asynchrony, which results in a generation of an alarm, is, for example, between 10% and 100%, in some embodiments between 10% and 50%. The alarm can be output, for example, via an interface, such as a display or a data connection for telemonitoring.

The detection of asynchrony can furthermore be used, for example, in order to identify unfavorable settings of the ventilator 1 and/or an intrinsic PEEP (positive end expiratory pressure). Unfavorable settings of the ventilator 1 can be found, for example, in pressure settings and/or flow settings and mean a non-optimal or inadequate respiratory treatment or assistance of the respiration of the living being.

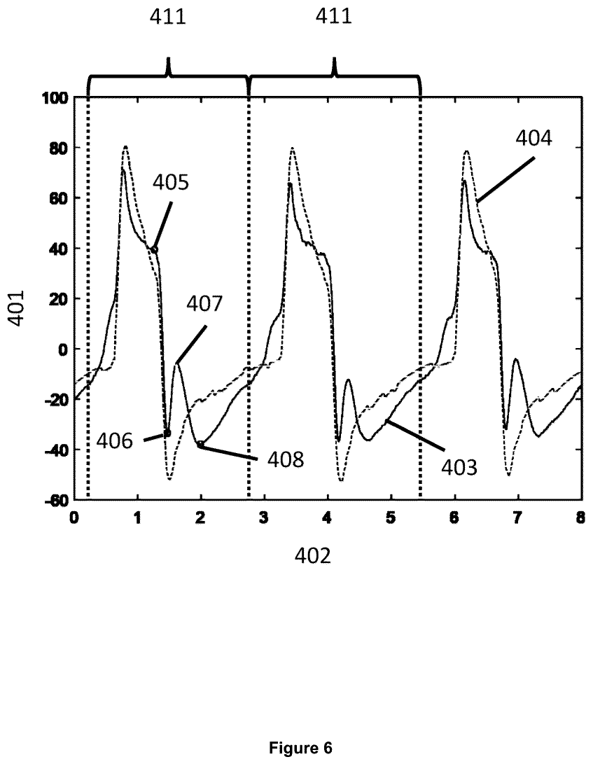

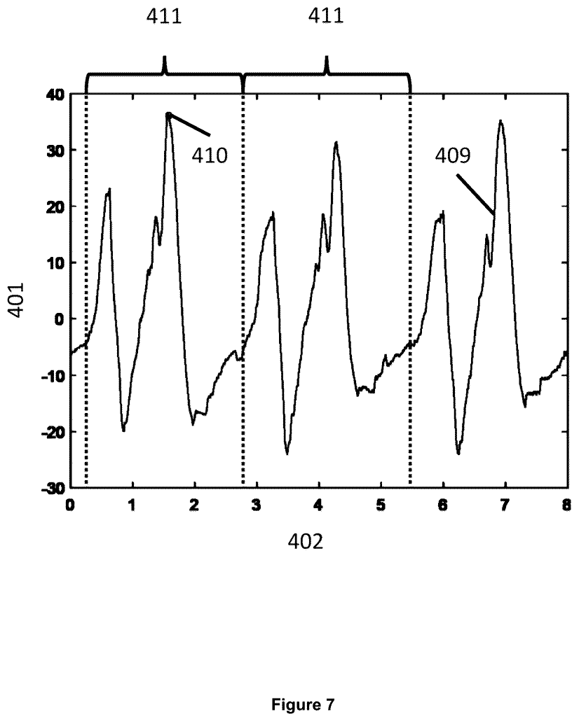

FIGS. 6 and 7 show by way of example the detection of early expiration triggers 412 via the respiratory exertion flow 409 or the determined respiratory flow 403 and the expected respiratory flow 404. In the diagram of FIG. 6, the flow rate 401 of the determined respiratory flow 403 and the expected respiratory flow 404 is plotted against the time 402. Two full breaths 411 are shown. The flow rate 401 strongly increases at the beginning of the inspiration and then gradually flattens out or sinks again at the end of the inspiration. From a specific flow rate 401, for example, a pressure ramp is started (beginning 405 of the pressure ramp), via which, for example, the pressure specified by the ventilator 1 is decreased in order to assist, enable, and/or induce the expiration of the patient. During an expiration, air escapes from the lung, a negative flow rate 401 is thus expected (expected respiratory flow 404) and also measured (measured respiratory flow 403). After the end 406 of the pressure ramp by which the respiratory gas pressure was reduced by the ventilator 1, a gradual flattening of the flow rate 401 is to be observed, which increases again from a negative peak (or a minimum) or approaches a flow rate 401 of 0. From a specific flow rate 401, for example, a pressure ramp is again started by the ventilator 1, via which it switches to an inspiratory pressure assistance.

In the case of an early expiration trigger 412, the pressure ramp on the expiratory pressure is started too early. The patient/user is not yet finished with the inspiration at the point in time, thus still requires air. After the end 406 of the pressure ramp, a further strong increase of the flow rate 401 of the determined respiratory flow 403 is therefore also to be detected up to a local maximum 407. The patient subsequently adapts to the expiration, thus exhales. As also without early expiration trigger 412, the flow rate 401 also passes through a local minimum 408 here before the flow rate 401 slowly increases again.

The detection unit 14, possibly in combination with the calculation unit 13 and/or the preparation unit 12 and/or further components of the ventilator 1, is configured, for example, to detect early expiration triggers on the basis of the course of the determined respiratory flow 403 and the expected respiratory flow 404.

For example, four points are checked in particular for this purpose: the start 405 of the pressure ramp from inspiration to expiration (by the ventilator 1), the end point 406 of the pressure ramp, the position of the local maximum 407 of the respiratory flow, which is not to be located further than in a time span between 0.3 seconds and 0.4 seconds after the end point 406 of the pressure ramp, and the local minimum 408, which is to follow the end point 406 of the pressure ramp within a time span of between 0.5 seconds and 0.7 seconds.

An early expiration trigger is detected in the exemplary embodiment, for example, if at least one of the following conditions applies:
1. The determined respiratory flow 403 is above 0 l/min at the beginning 405 of the pressure ramp
2. The determined respiratory flow 403 is below 0 l/min at the end 405 of the pressure ramp
3. e*determined respiratory flow 403 (at the local maximum 407)/determined respiratory flow 403 (at the end 406 of the pressure ramp)<expected respiratory flow 404 (maximum)/expected respiratory flow 403 (at the end of the pressure ramp)
4. f*determined respiratory flow 403 (at the local maximum 407)>determined respiratory flow 403 (at the end 406 of the pressure ramp)
5. g*determined respiratory flow 403 (at the local minimum 408)<determined respiratory flow 403 (at the end 406 of the pressure ramp)+determined respiratory flow 403 (at the local maximum 407)
6. (h<determined respiratory flow 403 (at the local maximum 407)−determined respiratory flow 403 (at the end 406 of the pressure ramp)) OR (h<determined respiratory flow 403 (local maximum 407)−determined respiratory flow 403 (at the local minimum 408))
7. Determined respiratory flow 403 (at the local minimum 408)<0

In this case the factors are e in a range from 0.5 to 2.0, preferably between 1 and 1.8; fin a range from 0.9 to 2.9, preferably in a range from 1.7 to 2.3; g in a range from 1 to 3, preferably between 1.8 and 2.2; and h in a range from 2 l/min to 101/min, preferably between 3 l/min and 7 l/min.

In some embodiments, it is provided that at least two or four or all of the conditions have to apply so that an early expiration trigger is detected.

An early expiration trigger 412 may also be detected in the course of the respiratory exertion flow 409, as shown in FIG. 7. The same breaths 411 can be seen as in FIG. 6, here as the course of the flow rate 401 of the respiratory exertion flow 409 with the time 402. Due to the local maximum 407 of the determined respiratory flow 403, a local maximum 410 can also be seen in the respiratory exertion flow 409. The expected respiratory flow 404 does not provide any direct increase of the flow rate 401 after the end 406 of the pressure ramp, therefore a large difference results here from the determined respiratory flow 403, which is reflected in the strongly positive local maximum 410 of the respiratory exertion flow 409. In some embodiments, for example, a statement about early expiration triggers can be made via the height of the local maximum 410 and also location in relation to the end 406 of the pressure ramp. For example, a change of the trigger sensitivity 3 can be controlled on the basis of the height of the local maximum 410. In some embodiments, for example, it is conceivable that the trigger sensitivity 3 is adapted on the basis of stored values for the height of the local maximum 410.

The system is designed in some embodiments to adapt the trigger sensitivity 3 on the basis of the early expiration triggers 412. For example, such an adaptation can take place automatically. If a certain number of early expiration triggers 412 is detected within a (possibly settable) period of time and/or a (possibly settable) number of breaths 411, the ventilator 1 can be configured, for example, so as to detect that an excessively sensitive trigger sensitivity 3 is provided for switching from inspiration to expiration. For example, the trigger sensitivity 3, at least for switching from inspiration to expiration, is then reduced, thus set to a less sensitive value.

The (automatic) setting of the trigger sensitivity 3 on the basis of missed breaths 218 and/or short trigger delays 305 and/or incorrect triggerings 307 relates in particular, for example, to a (threshold) value which relates to switching to an inspiration assistance (for example switching from expiration to inspiration). If early expiration triggers 412 are also incorporated in the setting, these thus relate, for example, in particular to (threshold) values, on the basis of which switching takes place from inspiration to expiration.

### LIST OF REFERENCE NUMERALS

1 ventilator
2 asynchrony
3 trigger sensitivity
4 automatic trigger sensitivity
11 sensor unit
12 preparation unit
13 calculation unit
14 detection unit
15 storage unit
16 monitoring unit
17 control unit
18 fan/valve unit
201 flow rate (flow) (y axis)
202 time (x axis)
203 determined respiratory flow
204 expected respiratory flow
205 expected trigger point
206 local maximum
207 expected respiratory flow
208 breath duration
209 respiratory exertion flow
210 difference (left)
211 difference (right)
212 difference (left)
213 difference (right)
214 minimum (left)
215 minimum (right)
216 breath
217 breath
218 missed breath
219 local maximum
301 flow rate (flow) (y axis)
302 time (x axis)
303 determined respiratory flow
304 expected respiratory flow
305 trigger delay
306 respiratory exertion flow
307 incorrect triggering
308 short trigger delay
309 breath
401 flow rate (flow) (y axis)
402 time (x axis)
403 determined respiratory flow
404 expected respiratory flow
405 starting point (pressure ramp)
406 end point (pressure ramp)
407 local maximum
408 local minimum
409 respiratory exertion flow
410 local maximum
411 breath
412 early expiration trigger

1.-49. (canceled)

50. A system for detecting asynchronies between a ventilator and a living being, wherein the system comprises at least one ventilator which comprises at least
a sensor unit,
a preparation unit,
a calculation unit,
a detection unit,
a storage unit,
a monitoring unit,
a control unit, and
a fan/valve unit;
and wherein the detection unit is configured to detect asynchronies between the ventilator and the living being based on respiratory parameters of the living being, and the control unit is configured to at least partially and least temporarily automatically control the ventilator based on the asynchronies detected by the detection unit.

51. The system of claim 50, wherein the detection unit detects missed breaths and/or short trigger delays and/or incorrect triggerings and/or premature expiration triggers and evaluates same as asynchronies.

52. The system of claim 51, wherein the detection unit detects the missed breaths and/or the short trigger delays by evaluating a respiratory exertion, an expected respiratory flow, and a determined respiratory flow.

53. The system of claim 52, wherein the calculation unit determines the respiratory exertion flow from the expected respiratory flow and the determined respiratory flow.

54. The system of claim 52, wherein the detection unit detects the missed breaths based on at least one of the following features of a respiratory exertion flow, an expected respiratory flow and/or a determined respiratory flow:
a local maximum of the determined respiratory flow lies between two minima of the respiratory exertion flow:
difference between a temporal position of a local maximus of the respiratory exertion flow and a corresponding left minimum:
difference between a temporal position of a local maximum of the respiratory exertion flow and a corresponding right, minimum;
difference between values of a local maximum of the respiratory exertion flow and a corresponding left minimum;
difference between values of a local maximum of the respiratory exertion flow and a corresponding right minimum;
expected respiratory flow at a point in time of a local maximum of the respiratory exertion flow;
time between a local maximum of the respiratory exertion flow and expected trigger point in time.

55. The system of claim 52, wherein the calculation unit determines an expected respiratory flow from an airway resistance R and a lung elasticity E.

56. The system of claim 55, wherein the calculation unit calculates the airway resistance R and the lung elasticity E from measured values measured by the sensor unit and prepared by the preparation unit.

57. The system of claim 55, wherein the airway resistance R and the lung elasticity E are determined by the calculation unit via a mathematical lung model.

58. The system of claim 55, wherein the airway resistance R and the lung elasticity E are determined by the calculation unit via a multiple linear regression and a one-compartment lung model.

59. The system of claim 51, wherein the control unit automatically sets a trigger sensitivity of the ventilator based on the missed breaths and short trigger delays detected by the detection unit.

60. The system of claim 59, wherein the control unit further automatically sets the trigger sensitivity of the ventilator based on incorrect triggerings detected by the detection unit.

61. The system of claim 59, wherein the control unit automatically adapts the trigger sensitivity in accordance with a number of detected short trigger delays and missed breaths within a time interval of from 0.5 minutes to 5 minutes.

62. The system of claim 59, wherein with leakage flows of greater than a threshold flow of from 15 l/min to 50 l/min, the trigger sensitivity is set in the form of an average trigger sensitivity, which is determined with incorporation of trigger sensitivities from prior periods of time having leakage flows below the threshold flow.

63. The system of claim 59, wherein the trigger sensitivity can be set manually and automatically, an automatic setting being able to set lower threshold values of parameters for triggering as is possible by a manual setting.

64. The system of claim 59, wherein the trigger sensitivity comprises a value which controls switching from an inspiration phase to an expiration phase, the ventilator being configured and designed to set the value of the trigger sensitivity that controls the switching from an inspiration phase to an expiration phase based on detected premature expiration triggers.

65. The system of claim 51, wherein a trigger delay is detected as a short trigger delay if the trigger delay is less than or equal to a threshold value, the threshold value being in a range of from 0 seconds to 0.5 seconds.

66. The system of claim 65, wherein the detection unit detects and determines the trigger delay via an offset between a respiratory exertion of the living being and a triggering of the ventilator.

67. The system of claim 51, wherein the ventilator is configured and designed to detect premature expiration triggers based on a time curve of a determined respiratory flow and/or an expected respiratory flow and/or a respiratory exertion flow.

68. The system of claim 51, wherein the ventilator is configured and designed to detect premature expiration triggers based on a position and value of a flow rate of a start of a pressure ramp, an end of a pressure ramp, a local maximum and/or a local minimum of a determined respiratory flow and/or an expected respiratory flow.

69. The system of claim 51, wherein the ventilator is configured and designed to detect premature expiration triggers if at least one of the following conditions is present:
determined respiratory flow is above 0 l/min at beginning of a pressure ramp;
determined respiratory flow is below 0 l/min at end of pressure ramp;
e*determined respiratory flow (at local maximum)/determined respiratory flow (at end of pressure ramp)<expected respiratory flow (maximum)/expected respiratory flow (at end of pressure ramp)
f*determined respiratory flow (at local maximum)>determined respiratory flow (at end of pressure ramp)
g*determined respiratory flow (at local minimum)<determined respiratory flow (at end of pressure ramp)+determined respiratory flow (at local maximum)
(h<determined respiratory flow (at local maximum)−determined respiratory flow (at end of pressure ramp)) OR (h<determined respiratory flow (at local maximum)−determined respiratory flow (at local minimum)
determined respiratory flow (at local minimum)<0;
e ranging from 0.5 to 2.0; f ranging from 0.9 to 2.9; g ranging from 1 to 3; and h ranging from 2 l/min to 10 l/min.
